(19) United States
(12) Patent Application Publication
(10) Pub. No.: US 2023/0309794 A1
(43) Pub. Date: Oct. 5, 2023

(54) ALBARRAN AND SHAFT FOR AN ALBARRAN

(71) Applicant: OLYMPUS WINTER & IBE GMBH, Hamburg (DE)

(72) Inventors: Kevin SCHULZ, Wilstedt (DE); Irina SCHMUCK, Heide (DE); Bastian SCHROEDER, Hamburg (DE); Andreas RUEHS, Ahrensburg (DE)

(73) Assignee: OLYMPUS WINTER & IBE GMBH, Hamburg (DE)

(21) Appl. No.: 18/124,934

(22) Filed: Mar. 22, 2023

Related U.S. Application Data
(60) Provisional application No. 63/325,374, filed on Mar. 30, 2022.

(51) Int. Cl. A61B 1/00

(52) U.S. Cl. CPC A61B 1/00098; A61B 1/00135

(57) ABSTRACT

A shaft and an Albarran, which can be produced in a particularly simple way and can be cleaned particularly easily and time-efficiently. This is achieved by a shaft, or an optical guide tube, and at least one pull wire tube guide being formed in one piece. Owing to this one-piece configuration of the shaft, the long closed pull wire tubes can be eliminated by design. Because of the open construction of the shaft, the shaft can be cleaned particularly easily and time-efficiently. Furthermore, the production of the shaft is particularly straightforward.

### DESCRIPTION

The invention relates to a shaft for an Albarran according to the preamble of claim 1. The invention furthermore relates to an Albarran as claimed in claim 10.

Albarrans are used to assist in operations or treatments with surgical instruments, for example endoscopes, resectoscopes, cystoscopes or the like. An Albarran may, for example, be used to deflect flexible forceps in the interior of a patient in a purposeful and controlled way. For this purpose, the Albarran, in the same way as for example an endoscope, comprises a shaft in the form of a rod or tube, which is to be guided with a distal end into the body of the patient. At a proximal end of the Albarran, outside the patient, the shaft is connected to a main body. Through this main body, further instruments or tools, for example an optical unit, wires or the like, can be guided via various openings, or ports, through the shaft into the patient.

At the distal end of the shaft, the Albarran comprises a lever, the so-called Albarran lever. This lever is configured to be movable and can be actuated, or pivoted, by means of a toggle on the main body. For this purpose, the lever is mechanically coupled to the toggle along the shaft. This mechanical coupling may involve either a rod or a pull wire. In general, the lever is connected to the toggle by means of two pull wires. It is conceivable for the two pull wires to be equally used for pivoting the lever to and fro, or for different movements of the lever to be achievable by means of the two pull wires via the toggle.

The pull wires are respectively guided from the proximal end to the distal end of the shaft through a pull wire tube. These pull wire tubes are aligned parallel to the shaft and are welded onto the outer wall of the shaft. The shaft, which may inter alia also be used for guiding the optical unit, is in this context also referred to as an optical guide tube. This optical guide tube, or the shaft, is substantially larger in its diameter than the two pull wire tubes.

Owing to the guiding of the pull wires inside the pull wire tubes, on the one hand the wires are guided and on the other hand bending of the wires during their transverse movement for actuating the lever is avoided.

In order to seal the main body, or the drive body, which is connected to the pull wire tubes, against any fluids, it is known to fill the thin pull wire tubes with grease. A sealing grease barrier is thereby established between the fluid distally and the main body proximally. This filling gives rise to long and thin grease-filled channels, or annular spaces, inside the pull wire tubes.

This known design of the shaft has several disadvantages. Because of the increased requirements, the long-term stability of the grease barrier in the pull wire tubes cannot always be ensured. A further disadvantage of the known shaft lies in the long weld seams between the pull wire tubes and the optical guide tube, since their production entails an increased cost outlay. Furthermore, it has been found that the long gaps between the pull wire tubes and the optical guide tube increase the cleaning time and the cleaning outlay. An improvement in this regard would lead to a product which is optimized for the customer.

On the basis of this, the object of the invention is to provide a shaft and an Albarran which can be produced in a particularly simple way and can be cleaned particularly easily and time-efficiently.

A solution to this object is described by the features of claim 1. It is accordingly provided that the shaft, or the optical guide tube, and at least one pull wire tube guide are formed in one piece. Owing to this one-piece configuration of the shaft, the long closed pull wire tubes can be eliminated by design. Because of the open construction according to the invention of the shaft, the shaft can be cleaned particularly easily and time-efficiently. Furthermore, the production of the shaft is particularly straightforward.

Preferably, the invention provides that the shaft is configured as a profiled tube, the at least one pull wire tube guide being a protrusion parallel to a longitudinal axis of the profiled tube, and the protrusion in this case constituting a cross-sectional widening of the, preferably concentric, profiled tube. In particular, it is provided that the profiled tube comprises two pull wire tube guides configured as protrusions, which are oriented parallel to one another to the longitudinal axis of the profiled tube, for receiving two pull wires. The profiled tube is configured in such a way that it is equally used to receive an instrument, in particular the optical unit, and the two pull wires. The pull wires are fixed in their protrusions by the optical unit during use of the shaft. Bending of the wires during the treatment can thereby be prevented. By removing the optical unit from the shaft after the end of the treatment, the pull wires are exposed and the open, and therefore easy to clean, design of the shaft is thereby achieved. Because this shaft no longer comprises long weld seams in the longitudinal direction, the production is particularly straightforward. Furthermore, long gaps, the cleaning of which is particularly time-intensive, are not formed.

According to a further preferred exemplary embodiment of the invention, a size, or a radius of curvature, or a shape of the at least one protrusion may correspond to, in particular be slightly greater than, a size, or a radius of curvature, or a shape of the at least one pull wire tube. The protrusions are dimensioned in such a way that the pull wires can be laid, or inserted, into these protrusions and can be moved transversely to and fro parallel to the longitudinal axis of the shaft during the treatment without a significant frictional or sliding resistance.

According to the invention, it is conceivable for the at least one protrusion to enclose the pull wire partially, preferably over an angle of from 45° to 270°, in particular over an angle of from 90° to 180°. Owing to this partial enclosure of the pull wires by the protrusions, the pull wires are already held, or fixed, in their position before the introduction of the optical unit. This may be helpful for simplifying the assembly of the shaft overall.

A further aspect according to the invention may consist in a tube segment respectively being arranged at a distal end and/or at a proximal end of the profiled tube. This at least one tube segment may be configured concentrically and have the same radius of curvature as the profiled tube. For a mechanical connection, it is conceivable for the tube segments to be welded with one of their end sides to the end sides of the distal and/or proximal ends of the profiled tube. This welding may be carried out end-to-end, and is therefore straightforward. At the proximal end of the shaft, the tube segment is used to engage in a firm connection to the, in particular conventional, main body. At the distal end of the shaft, the tube segment positioned there may be used to receive the Albarran lever and the fastening thereof. The tube segment, or the tube segments, are furthermore used to guide the pull wires. Specifically, the pull wires are guided along the outer wall of the tube segments, so that additional protection from bending may also be achieved.

Preferably, it is furthermore conceivable for the proximal and/or the distal end of the profiled tube or of the at least one tube segment to comprise an at least substantially concentric tongue, the diameter of which is less than the diameter of the profiled tube and of the tube segment. This tongue may be used to connect, or weld, the profiled tube to the at least one tube segment. By this tongue, the profiled tube and the at least one tube segment may be fitted together and then welded. The welding may therefore be carried out in a predefined and particularly straightforward way.

An Albarran for achieving the aforementioned object has the features of claim 10. Such an Albarran comprises an Albarran lever and a main body with a drive body. The Albarran lever can be moved via at least one pull wire by a toggle on the drive body. In this case, the at least one pull wire is mounted in at least one pull wire tube guide and an optical unit inside a tubular shaft as claimed in at least one of claims 1 to 9. Owing to the pull wire tube guide according to the invention, which is configured as a protrusion in a profiled tube of the shaft, on the one hand the Albarran can be cleaned particularly easily and rapidly and produced in a simple and therefore efficient way. By the interaction of an instrument, or the optical unit which is inserted into the profiled tube, and the pull wires, the latter can be protected against bending. After the optical unit is removed from the shaft and the pull wires are thereby exposed, the profiled tube, or the shaft, can be cleaned particularly time-efficiently and easily because of the open configuration. The Albarran described here, having the shaft according to the invention, has no weld seams which are aligned parallel to a longitudinal axis of the shaft, so that no long gaps or other regions which are difficult to access are formed. Owing to the open design of the shaft, the necessary cleanliness can also be achieved with little outlay. The Albarran claimed here is therefore particularly readily suitable for a multiplicity of reconditionings.

A preferred exemplary embodiment of the present invention will be explained in more detail below with the aid of the drawing, in which:

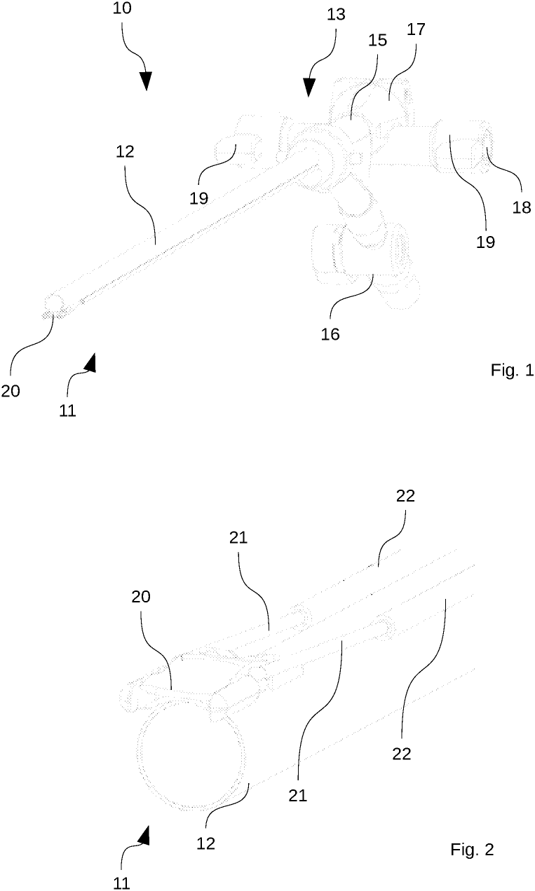

FIG. 1 shows a schematic representation of an Albarran according to the prior art,

FIG. 2 shows a view of a distal end of the Albarran according to FIG. 1,

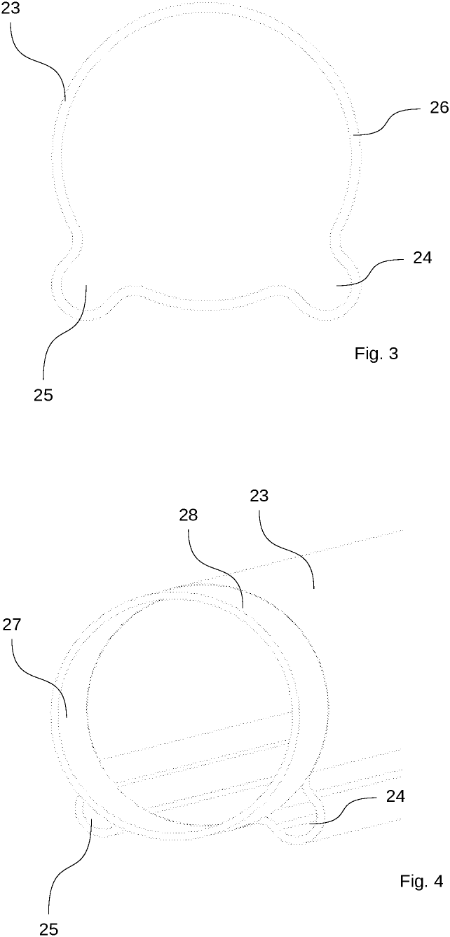

FIG. 3 shows a cross-sectional view of a shaft, and

FIG. 4 shows a perspective view of a distal end of the shaft.

FIG. 1 represents a known Albarran 10 according to the prior art. Here, only the features which are relevant for the description of the present invention will be indicated. For a description of all features and the functionality of the Albarran 10 according to FIG. 1, reference is made to the relevant prior art.

A main body 15 is arranged at a proximal end 13 of a shaft 12 of the Albarran 10. This main body 15 is directly connected or connectable to the shaft 12. While, for a treatment or operation on a patient, the elongate shaft 12 is guided with a distal end 11 into an interior of the body of the patient, the main body 15 remains outside the body and is used for providing and operating various tools or aids for carrying out the operation. For this purpose, for example, the main body 15 is assigned a tubular port 16 via which tools (not represented) or other aids can be guided during the operation through the shaft 12 to the distal end 11 of the Albarran 10 to the site of the operation.

Furthermore, the main body 15 comprises a drive body 17. A toggle axle 18 of a toggle 19 extends through this drive body 17 perpendicularly to a longitudinal axis of the Albarran 10, or of the shaft 12. By actuating this lever-like toggle 19, or by rotation of the toggle 19 about the toggle axle 18, an Albarran lever 20 at the distal end 11 of the Albarran 10 can be actuated. This Albarran lever 20 is used as an auxiliary tool during the operation, or the treatment. By actuating this lever 20, further tools (not represented) which have been introduced into the body through other surgical instruments, for example endoscopes or cystoscopes, may be assistively operated. By deflecting the Albarran lever 20 in the clockwise direction, for example, flexible forceps may be moved in the interior of the body.

For actuation of the Albarran lever 20, the drive body 17, or the toggle 19, is coupled in movement to one or two pull wires 21. It is known to guide these pull wires 21 outside the shaft 12. In this case, pull wires 21 are guided through in each case a tube segment 22, which are positioned on opposite sides of a wall of the shaft 12. For illustration, the shaft 12 with the Albarran lever 20 according to the prior art is represented in FIG. 2. This figure shows that the tube segments 22, also referred to as pull wire guides, extend parallel to the shaft 12 from the proximal end 13 to the distal end 11, or to the Albarran lever 20. There, the pull wire 21 is coupled to the Albarran lever 20 in such a way that an actuation of the lever 20 is possible. At a proximal end of the pull wires 21, they are connected in the drive body 17, or to a pull wire carrier.

By actuation of the toggle 19 about the toggle axle 18, the at least one pull wire 21 is tensioned so that the Albarran lever 20 rotates about a pivot axis. As soon as the toggle 19 is rotated in an opposite direction, the mechanical stress on the pull wire 21 is relaxed and the Albarran lever 20 moves back into its starting position. The toggles 19 are assigned to both sides of the drive body 17.

In contrast to the prior art, the invention provides that the pull wires 21 are guided inside a shaft 23 of the Albarran 10. FIG. 3 represents a cross section of the shaft 23 according to the invention. This shaft 23 is configured as a profiled tube having two protrusions 24, 25. These protrusions 24, 25 in a wall 26 of the shaft 23 are aligned parallel to a longitudinal axis of the shaft 23 and are used as a guide for the pull wires 21. The protrusions 24, 25 at least partially form a recess with a circular cross section. In this case, this circular cross section may enclose an angle of 45° - 270°, in particular 90° - 180°. In this way, the two pull wires 21 may be guided, or laid, through the two protrusions 24, 25 without great outlay. The pull wires 21 may be fixed inside the protrusions 24, 25 by using a further instrument, for example the optical unit, which is guided through the shaft 23. The further instrument in this case corresponds with its outer circumference to the inner diameter of the shaft. The pull wires 21 are thereby held in their protrusions 24, 25. Bending of the pull wires 21 is thereby prevented.

Owing to this open and one-piece configuration of the shaft 23 together with the protrusions 24, 25, or the pull wire guides, the Albarran 10 on the one hand may be assembled readily and on the other hand cleaned very easily and time-efficiently. Previously required greasing of the pull wire guides is also unnecessary with this profiled tube. After the end of the treatment, the instrument, or the optical unit, is withdrawn from the shaft 23 so that the two pull wires 21 may be exposed and may likewise be cleaned in a straightforward way. It is likewise conceivable for the shaft 23 simply to be decoupled from the main body 15 and withdrawn from the instrument, or the optical unit, and the pull wires 21.

In order to stabilize the pull wires 21 at the proximal and distal ends of the shaft 23, it is conceivable to fasten a tube segment 27 at these ends of the shaft 23. This tube segment 27 has the same radius of curvature as the shaft 23. The thickness of a wall 26 of the tube segment 27 is also preferably equal to the wall thickness of the shaft 23, so that the tube segment 27 joins seamlessly with the shaft 23 both on the external side and on the internal side. The tube segment 27, however, is configured concentrically and therefore does not comprise the protrusions 24, 25.

Preferably, it is provided that the one tube segment 27 or the two tube segments 27 are welded firmly at the ends of the shaft 23. This circumferential weld seam can be produced particularly easily and without great outlay. When the two pull wires 21 then emerge from the protrusions 24, 25 at the distal or proximal ends, they are guided along an outer wall 28 of the tube segments 27. By this guiding-along, the pull wires 21 are already fixed in the protrusions 24, 25. The additional instrument, or the optical unit, inside the shaft 23 then also serves to avoid the bending of the pull wires 21.

The tube segment 27 may vary in its length. In particular, it is conceivable for the tube segment 27 to be constituted in such a way that the lever can also be fastened on its outer wall 28. By virtue of the tube segments 27, the shaft 23 according to the invention also preserves its open and one-piece shape, which has proven particularly advantageous during production, during use and during cleaning.

Besides the exemplary embodiment presented here, it is also conceivable for the shaft 23 to comprise only one protrusion 24, 25, in which only one pull wire 21 is guided. Furthermore, it is also conceivable for the shaft 23 to comprise more than two protrusions 24, 25.

At the proximal end of the shaft 23, the pull wires 21 emerge from the protrusions 24, 25 and can be coupled as before to the main body, or the drive body. The pull wires 21 may also be used at the distal end of the shaft 23 with a lever as before, in order to move the latter for the intended use.

List of References

| | |
|---|---|
| 10 | Albarran |
| 11 | distal end |
| 12 | shaft |
| 13 | proximal end |
| 15 | main body |
| 16 | port |
| 17 | drive body |
| 18 | toggle axle |
| 19 | toggle |
| 20 | Albarran lever |
| 21 | pull wire |
| 22 | tube segment |
| 23 | shaft |
| 24 | protrusion |
| 25 | protrusion |
| 26 | wall |
| 27 | tube segment |
| 28 | outer wall |

1. A shaft for an Albarran, at the distal end of which an Albarran lever can be arranged and at the proximal end of which a main body with a drive body can be arranged, the Albarran lever being movable via at least one pull wire by a toggle on the drive body and the at least one pull wire being mounted in at least one pull wire guide and an optical unit being mounted inside the tubular shaft, wherein the shaft and the at least one pull wire guide are formed in one piece.

2. The shaft for an Albarran as claimed in claim 1, wherein the shaft is configured as a profiled tube, the at least one pull wire guide being a protrusion parallel to a longitudinal axis of the profiled tube, and the protrusion in this case constituting a cross-sectional widening of the profiled tube.

3. The shaft for an Albarran as claimed in claim 2, wherein the profiled tube comprises two pull wire guides configured as protrusions, which are oriented parallel to one another and to the longitudinal axis of the profiled tube, for receiving two pull wires.

4. The shaft for an Albarran as claimed in claim 2, wherein a size, or a radius of curvature, or a shape of the at least one protrusion corresponds to a size, or a radius of curvature, or a shape of the at least one pull wire.

5. The shaft for an Albarran as claimed in claim 2, wherein the at least one protrusion encloses the pull partially.

6. The shaft for an Albarran as claimed in claim 1, wherein a tube segment is respectively arranged at a distal end and/or at a proximal end of the profiled tube.

7. The shaft for an Albarran as claimed in claim 6, wherein the tube segment is configured concentrically and has the same radius of curvature as the profiled tube.

8. The shaft for an Albarran as claimed in claim 6, wherein the tube segment can be welded to the profiled tube.

9. The shaft for an Albarran as claimed in claim 6, wherein the proximal and/or the distal end of the profiled tube or of the at least one tube segment comprises an at least substantially concentric tongue, the diameter of which is less than the diameter of the profiled tube and of the tube segment.

10. An Albarran having an Albarran lever and a main body with a drive body, wherein the Albarran lever can be moved via at least one pull wire by a toggle on the drive body and the at least one pull wire is mounted in at least one pull wire guide and an optical unit is mounted inside a tubular shaft as claimed in claim 1.
